(19) United States
(12) Patent Application Publication
(10) Pub. No.: US 2026/0325488 A1
(43) Pub. Date: Oct. 22, 2026

(54) METHODS AND COMPOSITIONS FOR TREATING AUTISM USING S-ETHOSUXIMIDE

(71) Applicant: Charles E. Niesen, Pasadena, CA (US)

(72) Inventor: Charles E. Niesen, Pasadena, CA (US)

(21) Appl. No.: 19/539,983

(22) Filed: Feb. 13, 2026

Related U.S. Application Data
(60) Provisional application No. 63/758,916, filed on Feb. 14, 2025.

(51) Int. Cl. A61K 31/4015; A61P 25/00

(52) U.S. Cl. CPC A61K 31/4015; A61P 25/00

(57) ABSTRACT

Pharmacological methods and single unit dosage forms for treating the expressive language deficit in an autistic human child or adult are provided. A therapeutically effective dose of enantiomerically enriched S-ethosuximide or a pharmaceutically acceptable salt thereof is administered to a patient suffering from expressive language deficit, preferably over an extended period, e.g., six months or longer, to reduce the autistic patient's expressive language deficit.

This application claims the benefit of U.S. provisional patent application No. 63/758,916 filed Feb. 14, 2025, the entire contents of which are incorporated herein by this reference.

### BACKGROUND OF THE INVENTION

The invention relates to pharmacological methods, compositions, and single unit dosage forms for treating autism, especially the expressive language deficit exhibited by children and adults who suffer from autism.

The term “autism spectrum disorder” (ASD) is used by neurologists to describe a range of conditions whose main feature is poorly developed/limited social interaction. Conditions typically present by age 2 and are reported to affect 1 out of 44 children in the United States, and as many as 75 million children worldwide. Patients suffering from ASD may exhibit social awkwardness or even ignore and refuse to interact with others. Autism lies at one end of the spectrum and is characterized by the patient being uninterested in interacting with other people. They ignore their parents as well as their peers. Also distinct in this group is their expressive language/communication deficit. Most autistic patients develop little to no expressive language, remaining mute, poorly communicative, and isolated their whole lives. Their language is rarely spontaneous. The urge to communicate is missing. They may develop 2-3 word phrases, but these phrases are said only in response to questions and not out of a want or desire to talk.

For example, an autistic person may be able to answer the question “What did you do this weekend?” but then lack the ability to add to or carry on a conversation. They may answer, “Saw movie,” but will not pursue the conversation with “And what did you do?” or “I saw a good movie. Have you seen this movie?” Thus, the central language/communication disorder in autism is characterized by not only 1) paucity of expressive language, but 2) inability to converse, to have a mutual and reciprocal exchange of words. The term “autism” was coined by Dr. Kanner in 1945 to describe this unique set of language and behavior problems he encountered in his young patients. These children did not want to communicate. They preferred to be alone, away from others, autonomous, hence autism.

This language deficit in autism stands in contrast to the communication problems in other ASD patients, specifically Asperger syndrome and pervasive developmental disorder (PDD). Patients with Asperger syndrome do not have a language/communication disorder. They speak in full sentences which are easily understood. They can carry on a conversation. Their deficit is a social one. They prefer limited social interaction and are awkward and unfamiliar with simple social discourse. They tend to be smart, often with better skills and more interest in the hard sciences, such as engineering or computer science. Patients with PDD, on the other hand, tend to have attention and learning problems and are often at several grade levels below their peers. As such, they do not know how to have a conversation, even though they may want to and can talk in full sentences. They are socially awkward. In conversation, they talk mainly about their own interests, in great detail, without engaging or showing interest in the other person's feelings or activities. For example, they will talk about their interest in dinosaurs or a computer game to the exclusion of what the other person wants to say.

Patients with mental retardation are not considered part of the ASD group. They may have simple or limited expressive language, but they are interactive and interested in being with other people. They will engage, gesture, hug, and try to communicate with others, unlike autistic patients who ignore others, often refusing to acknowledge even their presence.

Individuals with ASD are generally at higher risk of developing epilepsy, but the relationship between ASD and epilepsy is complex. Although antiepileptic drugs (AEDs) such as valproate, lamotrigine, levetiracetam, and others have been used extensively to treat other disorders, both in neurology and psychiatry, the medical community has not recognized that AEDs could have a role to play in treating expressive language deficit in autistic patients.

There is currently no FDA-approved treatment for childhood autism. Few if any pharmacological interventions specifically target expressive language. A recent, significant exception is the use of racemic ethosuximide to treat expressive language deficit, as disclosed in U.S. Pat. No. 11,980,603 (Niesen). The present invention builds on that work and provides enantiomeric methods and pharmaceutical compositions for treating autism.

### SUMMARY OF THE INVENTION

The present invention provides pharmacological methods, compositions, and devices for treating expressive language deficit in an autistic human. In one aspect of the invention, a therapeutically effective amount of enantiomerically enriched S-ethosuximide—or a pharmaceutically acceptable salt thereof—is administered to the autistic human, preferably for an extended period of time. In one embodiment, the drug is administered daily, or twice daily, for at least one month, preferably at least three months, more preferably at least six months, to increase the person's expressiveness and ability to converse with others.

The invention also encompasses single unit dosage forms comprising an enantiomeric excess of S-ethosuximide, or a pharmaceutically acceptable salt thereof, together with one or more pharmaceutically acceptable excipients, e.g., carriers, diluents, processing aids, flavoring agents, coloring agents, and other agents used to prepare pharmaceutical compositions.

### DETAILED DESCRIPTION

### 1.1 Methods of Treatment Using S-Ethosuximide

In one aspect of the invention, a pharmacological method for treating the expressive language deficit in an autistic human child or adult is provided. A therapeutically effective dose of enantiomerically enriched S-ethosuximide—or a pharmaceutically acceptable salt thereof—is administered to the patient, preferably over an extended period of time, e.g., one month, two months, three months, six months, or longer, to treat the patient and reduce the patient's expressive language deficit.

The R- and S-enantiomers of ethosuximide have formulas (I) and (II), respectively:

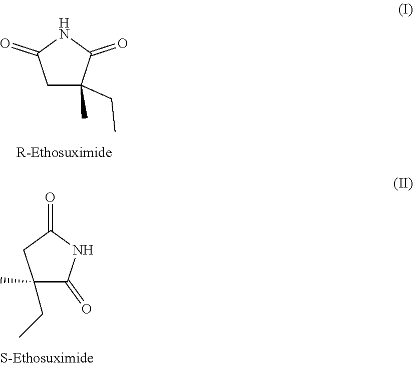

Ethosuximide is the drug of choice for treating childhood absence seizures. It is known to block T-type calcium channels and is not known to affect mood or behavior. Racemic ethosuximide—a 50-50 mixture of R and S isomers—is sold by Pfizer Inc. (NY, NY) under the brand name Zarontin®. It is available in capsule and syrup forms. Racemic ethosuximide can be made in a multi-step process starting with ethyl methyl ketone and ethyl cyanoacetate. The general scheme entails (1) Knoevenagel condensation of ethyl methyl ketone with ethyl cyanoacetate to yield ethyl-2-cyano-3-methyl-2-pentenoate, (2) addition of HCN, (3) hydrolysis, (4) decarboxylation, (5) amidization, and (6) deamination upon heating to close the ring and form ethosuximide. Chirality is introduced in step (2): nucleophilic addition of HCN, which can occur from above or below the double bond, resulting in a racemic mixture of R- and S-enantiomers.

The individual R-(−)- and S-(+)-enantiomers of ethosuximide can be prepared asymmetrically or isolated from the racemic ethosuximide using a chiral resolution technique. A synthetic route of interest is provided by Knabe and Koch (1972) and Knabe and Plisch (1973), who prepared a number of optically active disubstituted succinimides, including R- and S-ethosuximide. The overall scheme entails (1) preparation of racemic 3-methyl-3-ethyl-3-cyanopropionic acid from the corresponding racemic ethyl ester using methanolic KOH, acidification, isolation, and recrystallization from benzene/petroleum ether; (2) resolution using a chiral threo-base, e.g., threo-1-phenyl-2-aminopropanediol; and (3) conversion of the optically active ester amide obtained in step (2) into R- or S-ethosuximide using aqueous ammonia. Optical rotation data is reported as [α]_{D}20-32° and +32° (c, 3, chloroform) for R-(−)-ethosuximide and S-(+)-ethosuximide, respectively. See J. Knabe and W. Koch, “Synthese der Enantiomere einiger disubstituierter Succinimide,” [“Synthesis of the enantiomers of some disubstituted succinimides”], Archiv der Pharmazie, Vol. 305, pages 757-765 (1972); and J. Knabe, J Pilsch, “Die configuration von chiralen 2-aethyl-2-methyl-succinimiden” [“The configuration of chiral 2-ethyl-2-methyl-succinimides”], Tetrahedron Letters, Vol. 14, Issue 10, pages 745-746 (1973). Both papers are incorporated by reference herein in their entirety. Additional discussion and synthetic methodologies have been noted by Professor Janet Mifsud (now at the University of Malta) in her 1973 doctoral thesis (Queens University, Belfast).

For a pharmaceutical composition in which the active pharmaceutical ingredient (API) is a chiral compound having a single chiral center (i.e., ethosuximide), it can be convenient to describe the “enantiomeric purity” of the compound, that is, the extent to which a given sample or dose contains more of one enantiomer (S-ethosuximide) and less of the other, mirror image enantiomer (R-ethosuximide). Enantiomeric purity can be verified using nuclear magnetic resonance (NMR) spectroscopy and an appropriate NMR lanthanide shift reagent, e.g., Europium (III) tris [3-(trifluoromethylhydroxymethylene)-(+)-camphorate], available from Sigma-Aldrich.

A chiral resolution technique, such as capillary electrophoresis, more particularly micellar electrokinetic chromatography (MEKC), can also be used to separate the R and S enantiomers of ethosuximide and assess enantiomeric purity. An example is found in the Waters Application Library (Waters Corporation, Milford, MA; https: www.waters.com/webassets/cms/library/docs/970895.pdf) incorporated by reference herein. The instrumentation and conditions for this example are summarized in Table 1.

TABLE 1
Chiral MEKC Separation of Ethosuximide Enantiomers

| Compound/Type | Ethosuximide/enantiomeric mixture | |
|---|---|---|
| Matrix | Water | |
| Column/Capillary | AccuSep | |
| Column/Capillary Dimensions | 50 μm by 60 cm | |
| Column/Capillary Part No. | WAT250-01 | |
| Flow Rate/Voltage | 15 | KV |
| Temperature | 30 degrees C | |
| Injection Volume/Type | Hydrostatic | |
| Injection Conditions | 10 | seconds |
| Sample Concentration | 10 | μg/mL |
| Run Time | 17 | min. |
| Mobile Phase/Electrolyte | 25 mM, PO4/BO4, pH 8.0, 100 ml | |
| | Enantioselect-S-Val-1 | |
| Primary Detection wavelength | 214 | nm |

| | |
|---|---|
| Instrumentation/System | Waters Q4000E |
| | Millenium Chromatography Manager V2.1 |
| | Control |

From a therapeutic standpoint, it can be preferred to administer enantiomerically pure S-ethosuximide (substantially 100% S isomer) to a patient, as this ensures that substantially all of the ethosuximide is the enantiomer of choice. In practice, however, this may not always be possible. Thus, in the practice of the present invention, it can be acceptable—and more economical—to administer “enantiomerically enriched” S-ethosuximide, which may have less than 100% enantiomeric purity.

The term “enantiomeric excess” (ee) describes the absolute difference in mole fractions of the respective R and S enantiomers present in a sample:

ee=|F_{S}−F_{R}|, where F_{S}+F_{R}=1.

Enantiomeric excess can also be expressed as a percentage:

% ee=((|S−R|)/(S+R))×100%

where S and R denote the respective amounts of S and R isomers present. A sample that contains more S-ethosuximide than R-ethosuximide is enantiomerically enriched with the S isomer, as compared to racemic ethosuximide. Table 2 presents several nonlimiting examples.

TABLE 2
S-Ethosuximide Enantiomeric Enrichment

| Selection | Mole % S | Mole % R | % ee |
|---|---|---|---|
| 1 | 100 | 0 | 100 |
| 2 | 99 | 1 | 98 |
| 3 | 98 | 2 | 96 |
| 4 | 95 | 5 | 90 |
| 5 | 90 | 10 | 80 |
| 6 | 80 | 20 | 60 |

Accordingly, there are many embodiments of the invention, including without limitation those in which S-ethosuximide is administered to the patient in an enantiomeric excess (% ee) of at least 80%, at least 90%, at least 95%, at least 96%, or at least 98%. Enantiomeric purity can be expressed as the mole percent of the desired isomer (in this case, S-ethosuximide), or in terms of enantiomeric excess. However it is expressed, a high degree of enantiomeric purity is generally preferred from a therapeutic standpoint. For a single unit dosage form, as described herein, unless otherwise stated or defined, the term “enantiomerically enriched S-ethosuximide” means that at least 80% of the ethosuximide present in the composition is S-ethosuximide and no more than 20% of the ethosuximide is R-ethosuximide.

As an alternative to using S-ethosuximide, an enantiomeric excess of a pharmaceutically acceptable salt thereof is administered to the autistic patient, preferably over an extended period, e.g., 1-6 months or longer. A nonlimiting example is the acid addition salt, S-ethosuximide hydrochloride, prepared by treating S-ethosuximide with hydrochloric acid. More generally, any pharmaceutically acceptable salt of S-ethosuximide, can be utilized. Synthetic methods for preparing pharmaceutical salts are found in standard texts, such as the Handbook of Pharmaceutical Salts (P. Heinrich Stahl and Camille G. Wermuth, eds.), published jointly by Verlag Helvetica Chimica Acta (Zurich, Switzerland) and Wiley-VCH (Weinheim, Germany), 2008, chapter 11, pp. 249 et seq., incorporated by reference herein.

An autistic patient who exhibits expressive language deficit symptoms should be evaluated by a child neurologist before treatment begins, with particular attention given to the patient's language abilities and limitations. In one embodiment of the invention, an enantiomeric excess of S-ethosuximide, or a pharmaceutically acceptable salt thereof, is administered to the patient. The patient's language skills should be evaluated before treatment commences, and periodically during the course of treatment. It is preferred to administer the drug at least daily for at least one month, more preferably at least two months, more preferably at least three months, and even more preferably for six months or longer. The drug can be administered once a day, twice per day (b.i.d.), or even more frequently; however, a treatment regimen that calls for an autistic patient to receive medication more frequently may be met with compliance problems.

Drug dosage depends on the age and weight of the patient, with younger, lighter patients typically receiving a smaller dose than older children, teenagers, and adults. Dosage can be expressed as milligrams of drug per kilogram of body weight per day (mg/kg/day), that is, the number of milligrams of the active pharmaceutical ingredient, S-ethosuximide per kilogram of body weight per day. Dosage can also be expressed on an absolute basis (e.g., 60 mg/day). In one embodiment, S-ethosuximide is administered at a dosage of 50-1000 mg/day, depending on the age and/or weight of the patient. Dosage can also be expressed on a twice per day (b.i.d.) basis, e.g., 200 mg b.i.d. When the patient is a child, it can be helpful to set the dosage using the mg/kg/day methodology. For younger and/or smaller children, a suitable dosage could be 5-20 mg/kg/day. For teenagers and adults, a higher dosage range is likely to be more appropriate, e.g., 50-500 mg b.i.d. (100-1000 mg absolute), or perhaps more typically, 100-400 mg b.i.d. (200-800 mg of S-ethosuximide per day).

Two nonlimiting illustrations may be helpful: (1) a young patient weighing 441b (20 kg); dosage: (5 mg/kg/day)×20 kg=100 mg/day or 50 mg b.i.d. (2) a 15-year-old patient weighing 1101b (50 kg); dosage: 800 mg/day or 400 mg b.i.d.

### 1.2 Means of Evaluating Treatment Efficacy

To assess the effectiveness of S-ethosuximide as a pharmacological treatment for expressive language deficit, patients can be treated with S-ethosuximide for at least one month, more preferably six months or longer, and their language, social, and behavioral skills are monitored throughout the course of treatment, typically every 1-3 months. Study participants (subjects) are divided into four treatment groups based on neurodevelopmental level (mental status): normal IQ (NIQ), borderline IQ (BIQ), mild mental retardation (mMR), and moderate mental retardation (moMR). Language skills (expressiveness, receptivity), social behavior (social interaction, eye contact, participation), and mood (affect, irritability, agitation) were evaluated at various points in time. Verbal output is graded using a 7-point scale (0-6), where 0=nonverbal, 1=echolalic (the patient can respond by echoing the speaker), 2=single words (the patient can respond in single words), 3=phrases (the patient can respond in a simple phrase), 4=sentences (the patient can respond in a full sentence), 5=spontaneous speech (the patient can speak spontaneously), and 6=mutual speech (the patient can have a true conversation with another person). The 7-point scale for evaluating expressive language is summarized in Table 3.

TABLE 3
Expressive Language Scale

| Ability | Abbreviation | Numeric Rating |
|---|---|---|
| Mutual speech | MU | 6 |
| Spontaneous speech | SP | 5 |
| Sentences | SN | 4 |
| Phrases | PH | 3 |
| Single Words | SW | 2 |
| Echolalic | EC | 1 |
| Non-verbal | NV | 0 |

Social behavior and mood symptoms, based on those described in the Aberrant Behavior Checklist, an established behavioral rating system used in evaluating patients with intellectual disabilities (see Aman, M. G., et al., “The Aberrant Behavior Checklist: A behavior rating scale for the assessment of treatment effects,” American Journal of Mental Deficiency, 89 (5): 485-491 (1985), incorporated by reference herein), are also graded on a 7-point scale (0±3). Patients are assigned a zero value at baseline (score before treatment) and are given a positive score (+1 to +3) or negative score (−1 to −3) if their symptoms improve or worsen to a mild, moderate, or significant degree, respectively.

It is common for autistic patients to receive one or more medications as treatment for a variety of conditions and symptoms, including attention deficit/hyperactivity disorder, irritability, mood swings, anxiety, seizures, etc. Nonlimiting examples include aripiprazole, lacosamide, oxcarbazepine, gabapentin, lisdexamfetamine, clonidine, valproate, risperidone, and alprazolam. Subjects in the NIQ and BIQ groups are more likely to be taking fewer concomitant medications than subjects in the mMR and moMR groups.

To evaluate drug efficacy and onset of action, each patient is monitored for changes in language, social behavior, and mood at various times after commencement of ETS therapy. Performance characteristics expected to be observed for each function (expressive language, mood, behavior) are summarized in Table 4, at t=1 month after commencement of treatment and t=3 months after commencement of treatment.

TABLE 4
ETS Treatment - Target Observations of Onset of Action

| | Desired Observation after | |
|---|---|---|
| | commencement of treatment | |
| Function | After 1 month | After 3 months |
| Expressive language | Increased word count | 2-word phrases |
| | (talking more) | Listening better |
| | | makes wants clearer |
| Social behavior | Increased eye contact | Sits in groups |
| | More engaged | |
| Mood | Calmer (+1) | Fewer tantrums |
| | More focused (+1) | |

Ideally, the effect of S-ethosuximide on language acquisition over time should parallel or at least approach the time course of language development in normal (non-autistic) children. A non-autistic child, age 12-15 months, typically has a vocabulary of 4-6 words and can answer simple questions nonverbally. Spoken words may not always be clear. By age 18-23 months, vocabulary may increase to 50 words, and the child can use 2-word phrases and even combine words, such as “more milk.” From age 2-3 years, the child can use 3-word sentences.

Patients younger than 12 years old who are receiving S-ethosuximide can also be given speech and ABA (Applied Behavioral Analysis) therapy, e.g., speech therapy given twice a week for 30 minute sessions, while ABA therapy can be 5 days per week, 2-3 hours per day. ABA therapy is directed at training autistic patients to be more engaged in social settings and has less to do with spoken language. These two therapies are helpful because they provide patients an opportunity to practice language skills in a supportive environment to improve language and social skills. Their role cannot be underestimated, but it is not easily measured.

S-ethosuximide is expected to be well tolerated in all ages evaluated, just as racemic ethosuximide is. Common side effects reported in the literature for patients receiving racemic ethosuximide treatment for childhood absence epilepsy include abdominal distress, diarrhea, weight loss, hyperactivity, lethargy, leukopenia, and depression.

(Racemic) ethosuximide is known to block T-type calcium channels, but its mechanism of action in reducing the expressive language deficit in autistic patients remains undetermined. Without being bound by theory, it is possible to conjecture that S-ethosuximide acts on the brain's endocannabinoid system, or in novel networks that affect neurodevelopment.

Regardless of its mode of action, several things can be noted. Improvements in expressive language and social skills are expected in autistic patients even one month after commencing daily treatment with S-ethosuximide, and improvements should progress during treatment. The most gains are expected in patients who are treated for six months or longer, with the best results likely to be seen in younger patients and in patients with normal IQ, especially when ethosuximide treatment is coupled with language therapy, and/or applied behavioral analysis (ABA) therapy. Older children tend to have more behavioral problems and may not be receiving social therapy. Treatment with an enantiomeric excess of S-ethosuximide should also work in adults, especially if coupled with daily ABA therapy or similar therapy. Discontinuing treatment should not result in a loss of language gains.

S-ethosuximide drug therapy can be used to treat the expressive language deficit in autistic patients, including patients who are nonepileptic (not experiencing clinical seizures), patients who are epileptic (have experienced seizures), and patients who manifest epileptiform abnormalities on electroencephalograph (EEG) but have not experienced clinical seizures.

### 1.3 Pharmaceutical Compositions

In addition to methods of treatment, the invention also encompasses pharmaceutical compositions and devices containing enantiomerically enriched S-ethosuximide, such as single unit dosage forms suitable for oral, buccal, sublingual, mucosal, nasal, or transdermal administration to a patient. Nonlimiting examples of oral forms include tablets, capsules, liquids, oral dissolving forms, etc. In simplest terms, such compositions can be made by intimately admixing enantiomerically enriched S-ethosuximide with one or more excipients, as described herein.

S-ethosuximide is either sourced from an organic chemistry/medicinal chemistry contract research company, and/or synthesized as described herein (Knabe et al., supra).

Pharmaceutical compositions of the invention suitable for oral administration can be provided as discrete dosage forms, nonlimiting examples of which include tablets (chewable or non-chewable), caplets, capsules (such as soft elastic gelatin capsules), cachets, troches, lozenges, dispersions, suspensions, emulsions, solutions, elixirs, and other liquid dosage forms such as flavored syrups. Such dosage forms contain a predetermined amount of the active pharmaceutical ingredient (S-ethosuximide) and can be prepared by conventional methods of pharmacy, as described in Remington, The Science and Practice of Pharmacy, 23rd edition, Lippincott Williams & Wilkins—Publishers, Baltimore, MD, Philadelphia, PA (2020); cf. Remington's Pharmaceutical Sciences, 9th edition, Mack Publishing, Easton, PA (1990).

In one embodiment, enantiomerically enriched S-ethosuximide is combined in intimate admixture with at least one excipient according to conventional pharmaceutical compounding techniques. Nonlimiting examples of such excipients suitable for solid oral dosage forms (e.g., powders, tablets, capsules, caplets) include starches, sugars, micro-crystalline cellulose, diluents, granulating agents, lubricants, binders, and disintegrating agents. Nonlimiting examples of excipients suitable for use in oral liquid form include water, glycols, oils, alcohols, flavoring agents, preservatives, and coloring agents. Excipients should be fit for human consumption.

In general, pharmaceutical compositions of the invention are prepared by intimately admixing enantiomerically enriched S-ethosuximide with one or more liquid carriers, finely divided solid carriers, or both, processing aids and other excipients, and then (for solid dosage forms) shaping the product into the desired form, e.g., tablet, lozenge, etc. if necessary. For example, a tablet can be made by compression molding a mixture of free-flowing powders or granules containing the S-ethosuximide and a carrier/excipient(s) in a suitable machine. Prior to shaping, the pH can be adjusted if necessary.

Nonlimiting examples of excipients useful in making oral dosage forms of the invention include binders, lubricants, fillers, and disintegrants. Specific, nonlimiting examples include corn starch, potato starch, gelatin, acacia gum, sodium alginate, carboxy methyl cellulose and derivatives, calcium carbonate, dextrates, kaolin, mannitol, sorbitol, gelatinized starch, magnesium stearate, glycerin, edible oils. In some cases, the binder or filler is present in an amount of 50 to 99% by weight of the pharmaceutical composition or dosage form.

Oral dosage forms can be provided in controlled-release form (i.e., delayed release form) as described, for example in U.S. Pat. Nos. 3,845,770; 3,916,899; 3,536,809; 3,598,123; 4,008,719; 5,674,533; 5,059,595; 5,591,767; 5,120,548; 5,073, 543; 5,639,476; 5,354,556; 5,733,566, each incorporated by reference herein.

### 1.3.1 EXAMPLES

Examples 1 and 2 illustrate two different formulations for single usage dosage forms of pharmaceutical compositions containing S-ethosuximide to be prepared in accordance one aspect of the invention. The processing equipment, carriers, and excipients are described in U.S. Pat. No. 7,427,638 (col. 29-30), which is incorporated by reference herein in its entirety. That same patent contains additional description of other carriers, excipients, dosage forms, and processing equipment and techniques that can be used in the practice of the present invention, by substituting S-ethosuximide as the active pharmaceutical ingredient.

### Example 1. 200 mg Dosage Capsule

Table 5 presents batch and single dosage formulation for a 200 mg dose of S-ethosuximide in a size #0 capsule.

TABLE 5
Formulation for 200 mg Capsule

| | | Quantity | | Quantity |
|---|---|---|---|---|
| Material | % by Weight | (mg/capsule) | | (kg/batch) |
| S-ethosuximide | 40.00% | 200 | mg | 16.80 kg |
| Pregelatinized corn | 9.5% | 297.5 | mg | 24.99 kg |
| starch, NF5 | | | | |
| Magnesium stearate | 0.5% | 2.5 | mg | 0.21 kg |

| | | | |
|---|---|---|---|
| Total | 100.0% | 500 mg | 42.00 kg |

The corn starch (SPRESS B-820) and S-ethosuximide are passed through a screen (e.g., 710 μm), loaded into a diffusion blender, and blended for 15 minutes. The magnesium stearate is passed through a screen (e.g., 210 μm) and added to the mixer, and then the combined cornstarch, ethosuximide, and magnesium stearate are further blended. The resulting mixture is then encapsulated in a size #0 capsule (500 mg per capsule) using a Dosator-type capsule filling machine (8400 capsules/batch).

### Example 2. 100 mg Oral Dosage Tablet

Table 6 presents batch and single unit dosage formulation for a solid tablet containing a 100 mg dose of S-ethosuximide.

TABLE 6
Formulation for 100 mg Tablet

| | | Quantity | | Quantity | |
|---|---|---|---|---|---|
| Material | % by Weight | (mg/tablet) | | (kg/batch) | |
| S-ethosuximide | 40.00% | 100 | mg | 20.00 | kg |
| Microcrystalline | 53.5% | 133.75 | | 26.75 | |
| cellulose, NF | | | | | |
| Pluronic F-68 | 4.0% | 10.00 | | 2.00 | |
| surfactant | | | | | |
| Croscarmellose | 2.0% | 5.00 | | 1.00 | |
| sodium, type A, NF | | | | | |
| Magnesium stearate | 0.5% | 1.25 | mg | 0.25 | kg |
| Total | 100.0% | 250 | mg | 50.00 | kg |

Microcrystalline cellulose (an excipient), croscarmellose sodium (a disintegrating agent), and S-ethosuximide are passed through a #30 mesh screen. Pluronic F-68 surfactant (JRH Biosciences, Inc., Lenexa, KS) facilitates micelle formation/gelation and is passed through a #20 mesh screen. The surfactant and 0.5 kgs of croscarmellose sodium are loaded into a 16 qt. twin shell tumble blender and mixed for 5 minutes. The mix is then transferred to a 3 cubic foot twin shell tumble blender, the microcrystalline cellulose is added, and the contents of the blender are mixed for 5 minutes. The resulting blended mixture is passed through a roller compactor/hammer mill, then transferred back to the tumble blender. The remaining croscarmellose sodium and magnesium stearate (a lubricant and release agent) is added to the blender and the contents are blended for about 3 minutes. The final mixture is compressed on a rotary tablet press (200,000 tablet batch size, 250 mg/tablet).

A nonlimiting example of a liquid dosage form containing S-ethosuximide can be prepared by compounding S-ethosuximide with one or more carriers or excipients, preservatives, flavoring agents, antioxidants, etc. In one embodiment, a liquid dosage form of S-ethosuximide suitable for oral administration contains, per teaspoon (5 ml), 5-250 mg ethosuximide, USP; citric acid, anhydrous, USP; FD&C red No. 40; FD&C yellow No. 6; flavor; glycerin, USP; purified water, USP; saccharin sodium, USP; sodium benzoate, NF; sodium citrate, USP; and sucrose, NF. Such a formulation is prepared in a manner similar to that used to prepare Zarontin Oral Solution.

S-ethosuximide can also be administered by means of a transdermal patch. In one embodiment, a transdermal patch containing ethosuximide has the form of a small, circular disc, a square, or some other shape, typically ≤about 1″ in diameter or about 1″×1″. The patch includes a reservoir for S-ethosuximide, a selectively permeable membrane through which ethosuximide can pass into a patient's skin, an impermeable outer top layer, and a bottom layer or bottom circumferential layer of adhesive. The entire patch is flexible enough to be applied and removed from skin without breaking or tearing.

In one embodiment of the invention, a transdermal patch includes a reservoir containing S-ethosuximide (typically dissolved in water or other suitable solvent) and optionally one or more additional components, such as inert fillers, dyes, preservatives, antioxidants, processing aids, etc. An impermeable top layer protects the reservoir from above. The top layer may be plastic or plastic-backed foil and is impermeable to ethosuximide. A selectively permeable membrane sits directly beneath the reservoir. A layer of adhesive is affixed to the patch, at least along the circumference of the patch. A release liner is secured to the adhesive layer until the patch is ready to be affixed to a patient, at which point the release liner can be peeled away, thereby exposing the adhesive.

The reservoir containing S-ethosuximide can take various forms. In one embodiment, the reservoir is a simple cavity with sufficient volume to hold at least one dose of S-ethosuximide along with any other components. In an alternate embodiment, the reservoir is a solid or semi-solid plastic matrix infused with S-ethosuximide. In still another embodiment, the adhesive itself functions as both the reservoir for S-ethosuximide and as the selectively permeable membrane.

The selectively permeable membrane can be made of polyvinyl chloride, polystyrene, polyurethane, ethylene vinyl acetate, polyester, polyolefin, polycarbonate, or another suitable polymer. When the patch is affixed to a patient's skin, ethosuximide can diffuse through the selectively permeable membrane into the skin, at a controlled rate, and then into the bloodstream.

The adhesive used to mount the patch on the skin should have low allergenicity and be nonirritating to the skin. In addition, the adhesive should have good water resistance and cohesive strength. If the adhesive is located inwardly beyond the circumference of the patch (i.e., if it also covers all or part of the bottom center region), then it should be selected to be permeable to S-ethosuximide, to ensure that the drug can diffuse from the reservoir into the patient's skin. There are a number of medical grade, pressure-sensitive adhesives that can be used to construct the transdermal patch. Nonlimiting examples include acrylate ester/vinyl pyrrolidone copolymers, dimethyl silicone polymers, and acrylate polymers.

A general discussion of the construction and design of a transdermal patch is found at www.madehow.com, volume 3, in an article describing the nicotine treatment patch. The entire content of that article is incorporated by reference herein. Notably, ethosuximide is more water soluble than nicotine, and this should favor the manufacture and design of a transdermal patch for delivering S-ethosuximide to a patient. (Unlike ethosuximide, nicotine can attack or dissolve many of the materials used to make transdermal patch components, such as the adhesive and selectively permeable membrane.)

Upon reading this disclosure, other embodiments and modifications may be apparent to the skilled person. All such embodiments and modifications are encompassed by the invention, as delineated by the claims and equivalents thereof. The invention also can be characterized in one or more alternative legal/linguistic forms, perhaps more familiar to patent practitioners in other countries and regions. For example, the invention can be characterized as “a medicament having as its active pharmaceutical ingredient S-ethosuximide or a pharmaceutically acceptable salt thereof for use in the therapeutic treatment of autism” (or similarly, “ . . . for use in reducing an autistic human's expressive language deficit”). Such a medicament can be prepared as described herein by compounding the active pharmaceutical ingredient, S-ethosuximide, with one or more excipients. Alternatively, the invention can be characterized as “S-ethosuximide or a pharmaceutically acceptable salt thereof for use in the treatment of autism,” or “use of S-ethosuximide or a pharmaceutically acceptable salt thereof in the manufacture of a medicament for the treatment of autism.” These and similar legal/linguistic forms are encompassed within the scope of the invention.

What is claimed is:

1. A method of treating expressive language deficit in an autistic human, comprising:
administering a therapeutically effective amount of enantiomerically enriched S-ethosuximide to an autistic human to thereby reduce the autistic human's expressive language deficit.

2. The method of claim 1, wherein the therapeutically effective amount is 3-20 mg/kg/day.

3. The method of claim 1, wherein the therapeutically effective amount is 50-500 mg b.i.d.

4. The method of claim 3, wherein the therapeutically effective amount is 100-400 mg b.i.d.

5. The method of claim 1, wherein the therapeutically effective amount is 100-1000 mg/day.

6. The method of claim 1, wherein the ethosuximide is administered twice per day.

7. The method of claim 1, wherein the ethosuximide is administered daily for at least six months.

8. The method of claim 1, wherein the S-ethosuximide is administered orally.

9. A method of treating expressive language deficit in an autistic human, comprising:
administering a therapeutically effective amount of S-ethosuximide or a pharmaceutically acceptable salt thereof to an autistic human.

10. The method of claim 9, wherein the S-ethosuximide or a pharmaceutically acceptable salt thereof is administered daily.

11. The method of claim 9, wherein the S-ethosuximide or a pharmaceutically acceptable salt thereof is administered daily for at least six months.

12. A single unit dosage form which comprises about 50 mg to about 1000 mg of enantiomerically enriched S-ethosuximide or a pharmaceutically acceptable salt thereof; and a pharmaceutically acceptable carrier, excipient or diluent.

13. The dosage form of claim 12 wherein said dosage form is suitable for transdermal, mucosal, nasal, buccal, sublingual, or oral administration to a patient.

14. The dosage form of claim 12 wherein said dosage form is a capsule or a tablet.

15. The dosage form of claim 12 wherein the amount of enantiomerically enriched S-ethosuximide is from about 200 mg to about 800 mg.
